(19) United States
(12) Patent Application Publication
(10) Pub. No.: US 2024/0060098 A1
(43) Pub. Date: Feb. 22, 2024

(54) AMYCOLATOPSIS STRAINS FOR VANILLIN PRODUCTION WITH SUPPRESSED VANILLIC ACID FORMATION

(71) Applicant: BASF SE, Ludwigshafen am Rhein (DE)

(72) Inventors: Nicholas A. LYONS, Cambridge, MA (US); Rui ZHOU, Acton, MA (US); David NUNN, Lowell, MA (US)

(21) Appl. No.: 18/267,483

(22) PCT Filed: Dec. 17, 2021
(86) PCT No.: PCT/US2021/064099

§ 371 Date: Jun. 15, 2023

Related U.S. Application Data
(60) Provisional application No. 63/127,519, filed on Dec. 18, 2020.

(51) Int. Cl. C12P 7/24; C12N 9/02; C12N 9/06

(52) U.S. Cl. CPC C12P 7/24; C12N 9/0008; C12N 9/0016; C12Y 102/01067; C12Y 104/01013

(57) ABSTRACT

The present invention discloses mutant strains of Amycolatopsis sp. ATCC 39116 suitable for the production of natural vanillin using ferulic acid as a feedstock. More specifically, the present invention discloses mutant strains having mutations that reduce the degradation of vanillin to vanillic acid.

### CROSS REFERENCE TO RELATED APPLICATIONS

This application claims the benefit of U.S. Provisional Patent Application No. 63/127,519, filed Dec. 18, 2020, which is imported herein by reference in its entirety.

### SEQUENCE LISTING

The instant application contains a Sequence Listing that has been submitted electronically in ASCII format and is hereby incorporated by reference in its entirety. Said ASCII copy, created on Dec. 14, 2021, is named 074008_2039_00_WO_000325_SL.txt and is 29,167 bytes in size.

### FIELD OF THE INVENTION

The present disclosure generally relates to non-genetically modified microorganisms suitable for the production of natural vanillin using a renewable feedstock. More specifically, the present invention discloses Amycolatopsis strains having mutations that affect their vanillin degradation pathway, hence such strains are able to produce vanillin at increased yields.

### BACKGROUND OF THE INVENTION

Vanilla flavors are among the most frequently used flavors worldwide. They are used in the flavorings of numerous foods such as ice cream, dairy products, desserts, confectionary, bakery products and spirits. They are also used in perfumes, pharmaceuticals and personal hygiene products.

Natural vanilla flavor has been obtained traditionally from the fermented pods of vanilla orchids (Vanilla planifolia). It is formed mainly after the harvest during several weeks of a drying and fermentation process of the beans by hydrolysis of vanillin glucoside that is present in the beans. The essential aromatic substance of vanilla flavor is vanillin (4-hydroxy 3-methoxybenzaldehyde).

About 12,000 tons of vanillin are consumed annually, of which only 20-50 tons are extracted from vanilla beans. The rest is produced synthetically, mostly from petrochemical-derived guaiacol. In recent years, there is a growing interest in producing vanillin through biological fermentation using renewable feedstocks such as ferulic acid derived from rice bran, coniferyl alcohol from Spruce tree lignin, corn sugar and eugenol from clove oil. Vanillin derived from renewable feedstocks using biological fermentation is recognized as “natural vanillin” by regulatory and legislative authorities and can be marketed as “natural products”.

The actinomycete Amycolatopsis sp. strain ATCC 39116 has been in use for the bioconversion of ferulic acid to vanillin. This microorganism is known to metabolize ferulic acid using four major pathways distinguished by the initial reaction, namely nonoxidative decarboxylation, side chain reduction, coenzyme A-independent deacetylation, and coenzyme-A-dependent deacetylation. The coenzyme A-dependent deacetylation pathway for ferulic acid metabolism within the Amycolatopsis sp. strain ATCC 39116 has two steps. In the first step, ferulic acid is subjected to non-oxidative deacetylation to yield vanillin (FIG. 1). This step is mediated by two enzymes, namely, feruloyl-coenzyme A (CoA) synthetase coded by fcs gene and enoyl-CoA hydratase/aldolase coded by ech gene. Both these genes (ech and fcs) are within a single operon and the expression of these two genes is suppressed when Amycolatopsis sp. is grown in a culture medium containing glucose as the source of carbon. It is only after the addition of ferulic acid that the transcription of these genes is induced. As a result, when ferulic acid is added to the culture medium, there is a lag period of about 5 hours or more before vanillin synthesis could be detected. Typically, the lag period is at least 4 hours and as many as 8 hours. Once vanillin starts accumulating, the second stage of ferulic acid metabolism kicks in. In the second step, vanillin is subjected to β-oxidation to produce vanillic acid. The conversion of vanillin to vanillic acid is mediated by vanillin dehydrogenase enzyme coded by vdh gene. To increase vanillin production, there is a need to genetically engineer Amycolatopsis sp. such that the pathway responsible for degrading vanillin to vanillic acid is blocked.

### SUMMARY OF THE INVENTION

The present invention provides microorganisms having mutations that affect their vanillin degradation pathway. In a preferred embodiment of the present invention, the microorganism can be of the order Actinomycetales and the genus Amycolatopsis. For example, the microorganism can be a strain of Amycolatopsis sp. strain accessible under number ATCC 39116. One major challenge associated with the use of Amycolatopsis sp. ATCC 39116 for the bioconversion of ferulic acid to vanillin is the concurrent degradation of vanillin to vanillic acid by the vanillin dehydrogenase Vdh encoded by the vdh gene. As a result of the action of vanillin dehydrogenase on vanillin, the yield for vanillin obtainable from wild-type Amycolatopsis sp. ATCC 39116 is limited. Unlike its wild-type counterpart, the Amycolatopsis sp. strains developed according to the present invention show significantly reduced degradation of vanillin to vanillic acid. The mutant Amycolatopsis sp. ATCC 39116 strains of the present invention were obtained using chemical mutagenesis and the nature of the genetic mutations conferring the phenotype of reduced vanillin degradation to vanillic acid was identified by using whole genome sequencing. Significantly, the mutant strains according to the present invention are obtained without deleting or inactivating the vdh gene responsible for coding vanillin dehydrogenase which converts vanillin to vanillic acid.

One objective of the present invention is to identify mutant strains having genetic mutations that affect the vanillin degradation pathway. Because degradation of vanillin creates a source of energy for Amycolatopsis sp. cell, screening for mutants that can no longer grow when vanillin is the sole carbon source would uncover mutations that affect the vanillin degradation pathway. In addition, because vanillyl alcohol is converted to vanillin by Amycolatopsis sp. cells and vanillyl alcohol is less toxic to Amycolatopsis sp. cells than vanillin, the screening also can be performed by using vanillyl alcohol instead of vanillin as the sole carbon source.

Accordingly, in one aspect, the present invention provides a method for selecting mutant strains of Amycolatopsis sp. that exhibit reduced degradation of vanillin to vanillic acid compared to wild-type strains of Amycolatopsis sp. According to the method of the present invention, spores of Amycolatopsis sp. ATCC 39116 are exposed to a chemical mutagenic agent (e.g., methyl methanesulfonate) and grown in a liquid medium containing either vanillin or vanillyl alcohol as the sole carbon source. In addition, the present screening method also comprises adding an antibiotic such as penicillin to the liquid medium (what is known as the penicillin enrichment technique). The penicillin enrichment technique takes advantage of the fact that penicillin can kill only growing cells by inhibiting the cross-linking of peptidoglycan polymers essential for the structural integrity of the cell wall. Because Amycolatopsis sp. cells with a mutation in the vanillic acid utilization pathway will not be able to grow and undergo cell division in a growth medium containing vanillin or vanillyl alcohol as the sole source of energy, they will survive the penicillin treatment. Meanwhile, wild-type Amycolatopsis sp. cells that are growing and feeding on vanillin or vanillyl alcohol are killed by the penicillin.

Mutant strains identified according to the present invention show significantly less vanillic acid accumulation compared to the wild-type strains. In one embodiment, a mutant strain of Amycolaptosis sp. according to the present invention can show accumulation of less than 0.5 g of vanillic acid per liter of growth medium at more than 24 hours after ferulic acid is initially fed to the mutant strain. In another embodiment, a mutant strain of Amycolaptosis sp. according to the present invention can show accumulation of less than 0.25 g of vanillic acid per liter of growth medium at more than 24 hours after ferulic acid is initially fed to the mutant strain. In yet another embodiment, a mutant strain of Amycolaptosis sp. according to the present invention can show accumulation of less than 0.5 g of vanillic acid per liter of growth medium at more than 44 hours after ferulic acid is initially fed to the mutant strain. In another embodiment, the present mutant strain can show accumulation of less than 0.25 g of vanillic acid per liter of growth medium at more than 44 hours after ferulic acid is initially fed to the mutant strain.

In another aspect, the present invention provides a strain of Amycolatopsis sp. with a mutation in the gltBD operon. The inventors, through whole genome sequencing, have unexpectedly found that mutations in the gltBD operon could lead to defects in the vanillic acid utilization pathway, and hence a phenotype of reduced degradation of vanillin to vanillic acid. In some embodiments, a mutant strain of Amycolatopsis sp. according to the present teachings can comprise one or more mutation(s) in the gltB gene which comprises the nucleic acid sequence of SEQ ID NO: 1. In some embodiments, the present invention provides a strain of Amycolatopsis sp. with one or more mutations in the gltD gene comprising the nucleic acid sequence of SEQ ID NO: 3. The one or more mutations in the gltBD operon or the gltB gene or the gltD gene can be selected from the group consisting of a deletion, an insertion, a frameshift mutation, a promoter mutation, a missense mutation, a nonsense mutation, a slicing mutation, a point mutation, and any combinations thereof. According to the present invention, any one of the mutations in the gltBD operon or the gltB gene or the gltD gene selected from the group consisting of a deletion, an insertion, a frameshift mutation, a promoter mutation, a missense mutation, a nonsense mutation, a slicing mutation, a point mutation, and any combinations thereof can cause functional inactivation of the vanillic acid utilization pathway, which leads to reduced degradation of vanillin to vanillic acid.

In another aspect, the present invention provides a method for improving vanillin production using ferulic acid as a feedstock in a strain of Amycolatopsis sp. ATCC 39116. The method comprises causing one or more mutations in a strain of Amycolatopsis sp. ATCC 39116; specifically, in an endogenous gene responsible for regulating vanillin degradation into vanillic acid. The one or more mutations can be located within the endogenous gltBD operon, the gltB gene which comprises the nucleic acid sequence of SEQ ID NO: 1, the gltD gene comprising the nucleic acid sequence of SEQ ID NO: 3, or any combinations thereof. In preferred embodiments, the method also can comprise causing one or more mutations in an endogenous gene responsible for regulating the catabolism of ferulic acid to vanillin. For example, the one or more mutations can be located in an endogenous gene comprising the nucleic acid sequence of SEQ ID NO: 5 (the echR gene). As described in a co-pending application, the inventors unexpectedly have found that one or more mutations in the endogenous gene comprising the nucleic acid sequence of SEQ ID NO: 5 can lead to the functional inactivation of a protein comprising the amino acid sequence of SEQ ID NO: 6 (the EchR protein) which functions as a repressor of the ech-fcs operon. The ech-fcs operon regulates genes coding for the feruloyl-coenzyme A (CoA) synthetase and enoyl-CoA hydratase/aldolase enzymes which are responsible for the bioconversion of ferulic acid to vanillin. By inactivating the EchR protein, the transcription of the genes coding for the Ech and Fcs proteins are no longer repressed. As a result, vanillin is produced almost immediately as soon as ferulic acid is fed to the strain without the normally observed lag period of 4 hours or more. Accordingly, in one aspect, the present invention further provides a mutant strain of Amycolatopsis sp. having a first mutation in the gltBD operon and a second mutation in the echR gene comprising nucleic acid sequence of SEQ ID NO: 5. The mutations in the gltBD operon and the echR gene independently can be selected from the group consisting of a deletion, an insertion, a frameshift mutation, a promoter mutation, a missense mutation, a nonsense mutation, a slicing mutation, a point mutation, and any combinations thereof. In any of the foregoing embodiments, any of the mutations described can be caused by subjecting spores of the Amycolatopsis sp. ATCC 39116 strain to chemical mutagenesis. In alternative embodiments, at least one of the mutations in the gltBD operon and the echR gene can be caused by marker less genetic modifications using CRISPR technology or any other suitable recombinant DNA technology so that the gltB gene, the gltD gene and/or the echR gene are no more functional within the Amycolatopsis sp. cell. In certain embodiments, at least one of the mutations in the gltB gene, the gltD gene and/or the echR gene can be caused by chemical mutagenesis and at least one of the mutations in the gltB gene, the gltD gene and/or the echR gene can be caused by gene editing.

In one aspect, the present disclosure relates to a method of producing vanillin using a mutant strain of Amycolatopsis sp. described herein. In preferred embodiments, the mutant strain used comprises one or more mutations in the gltBD operon and one or more mutations in the echR gene. In certain embodiments, the mutant strain used in the present method for the production of vanillin using ferulic acid as a feedstock has no exogenous nucleic acid molecules and therefore can be qualified as a non-genetically modified organism (non-GMO). In some embodiments, the method of producing vanillin from ferulic acid can include (i) cultivating the mutant strain in a medium; (ii) adding ferulic acid to the medium to begin the bioconversion of ferulic acid to vanillin; and (iii) extracting vanillin from the medium.

The bioconversion method described herein can include recovering the vanillin from the mixture. The recovery of vanillin can be performed according to any conventional isolation or purification methodology known in the art. Prior to the recovery of the vanillin, the method also can include removing the biomass (enzymes, cell materials etc.) from the fermentation mixture.

Vanillin produced using the methods and/or the isolated recombinant host cells described herein can be collected, purified, and incorporated into a number of consumable product. For example, the vanillin can be admixed with the consumable product. In some embodiments, the vanillin can be incorporated into the consumable product in an amount sufficient to impart, modify, boost or enhance a desirable taste, flavor, or sensation, or to conceal, modify, or minimize an undesirable taste, flavor or sensation, in the consumable product. The consumable product, for example, can be selected from the group consisting of food, food ingredients, food additives, beverages, drugs and tobacco. The consumable product, for example, can be selected from the group consisting of fragrances, cosmetics, toiletries, home and body care, detergents, repellents, fertilizers, air fresheners, and soaps.

A first embodiment, a non-GMO mutant strain of Amycolaptosis sp., comprising: mutant strain of Amycolaptosis sp., formed by exposing a strain of Amycolaptosis sp., capable of producing vanillin and of metabolizing vanillin to vanillic acid to at least one mutagen, wherein said mutant strain of Amycolaptosis sp., is capable of producing vanillin and exhibits less degradation of vanillin to vanillic acid than does the strain, as measured by the accumulation level of vanillic acid in said mutant strain versus in the strain, in some embodiments the mutant strain is non-naturally occurring.

A second embodiment, a strain according to the first embodiment, wherein the mutant strain of Amycolaptosis sp., is a mutant of ATCC 39116.

A third embodiment, a strain according to the first and second embodiments, wherein said mutant strain of Amycolaptosis sp., accumulates less than 0.5 grams of vanillic acid per liter of the culture medium at more than 24 hours after ferulic acid is initially fed to the mutant strain.

A fourth embodiment, a strain according to the first and second embodiments, wherein said mutant strain of Amycolaptosis sp., accumulates less than 0.25 grams of vanillic acid per liter culture medium at more than 24 hours after ferulic acid is initially fed to the mutant strain.

A fifth embodiment, a strain according to the first and second embodiments, wherein said mutant strain of Amycolaptosis sp., accumulates less than 0.5 grams of vanillic acid per liter culture medium at more than 44 hours after ferulic acid is initially fed to the mutant strain.

A sixth embodiment, a strain according to the first and second embodiments, wherein said mutant strain of Amycolaptosis sp., accumulates less than 0.25 grams of vanillic acid per liter culture medium at more than 44 hours after ferulic acid is initially fed to the mutant strain.

A seventh embodiment, a mutant strain of the first through the sixth embodiments, wherein the genome of said mutant strain of Amycolaptosis sp., comprises one or more mutations in the gltBD operon.

An eighth embodiment, a mutant strain of the seventh embodiment, wherein the one or more mutations in the gltBD operon include at least one mutation in the gltB gene.

A ninth embodiment, a mutant strain of the eighth embodiment, wherein the one or more mutations are in the gltB gene comprise a nucleic acid sequence having at least 90 percent identity to SEQ ID NO: 1.

A tenth embodiment, a mutant strain of the eighth embodiment, wherein the one or more mutations are in the gltB gene comprising SEQ ID NO: 1.

An eleventh embodiment, a mutant strain of the seventh through the tenth embodiments, wherein the one or more mutations in the gltBD operon are in the gltD gene comprising a nucleic acid sequence having at least 90 percent identity to SEQ ID NO: 3.

A twelfth embodiment, a mutant strain of the seventh through the tenth embodiments, wherein the one or more mutations in the gltBD operon are in the gltD gene comprising nucleic acid sequence SEQ ID NO: 3.

A thirteenth embodiment, a mutant strain of the seventh through the twelfth embodiments, further including a mutation in an endogenous gene echR comprising the nucleic acid sequence SEQ ID NO: 5.

A fourteenth embodiment, a mutant strain of any one of the seventh through the thirteenth embodiments, wherein said one or more mutations are at least one mutation selected from the group consisting of a deletion, an insertion, a frameshift mutation, a missense mutation, a nonsense mutation, a slicing mutation, and a point mutation.

A fifteenth embodiment, a mutant strain of the fourteenth embodiment, wherein said mutation is a frameshift mutation comprising a 2 bp insertion.

A sixteenth embodiment, a mutant strain of the first through the fifteenth embodiments, wherein the mutant strain is obtained without permanently modifying the mutant strain by introducing any exogenous genetic material.

A seventeenth embodiment, wherein the mutant strain is obtained by contacting a strain with the mutagen methyl methanesulfonate.

An eighteenth embodiment, a process for producing vanillin, comprising the steps of: culturing the mutant strain according to any one of the first through the sixteenth embodiments in an appropriate medium comprising a substrate; and recovering the produced vanillin.

A nineteenth embodiment, a method for producing vanillin comprising: culturing a mutant strain of Amycolaptosis sp., according to any one of the first through the eighteenth embodiment in a medium containing a carbon source; and feeding ferulic acid to the mutant strain for enough time to allow conversion of ferulic acid to vanillin.

A twentieth embodiment, a strain of Amycolaptosis sp., comprising: a non-naturally occurring strain of Amycolaptosis sp., that includes a gene encoding vanillin dehydrogenase and at least one mutation in the gltBD operon, wherein said strain of Amycolaptosis sp., converts less vanillin to vanillic acid than does a strain of Amycolaptosis sp., without said at last one mutation in gltBD operon.

A twenty-first embodiment, a strain according to the twentieth embodiment, wherein the one or more mutations in the gltBD operon include at least one mutation in the gltB gene.

A twenty-second embodiment, a strain according to the twenty-first embodiment, wherein the one or more mutations are in the gltB gene comprise a nucleic acid sequence having at least 90 percent identity to SEQ ID NO: 1.

A twenty-third embodiment, a strain according to the twenty-first embodiment, wherein the one or more mutations are in the gltB gene comprising SEQ ID NO: 1.

A twenty-fourth embodiment, a strain according to the twentieth embodiment, wherein the one or more mutations in the gltBD operon are in the gltD gene.

A twenty-fifth embodiment, a strain according to the twenty-fourth embodiment, wherein the one or more mutations in the gltD gene comprises a nucleic acid sequence having at least 90 percent identity to SEQ ID NO: 3.

A twenty-sixth embodiment, a strain according to the twenty-fourth embodiment, wherein the one or more mutations in the gltD gene comprises nucleic acid sequence SEQ ID NO: 3.

A twenty-seventh embodiment, a strain according to the twentieth through the twenty-sixth embodiments, further including a mutation in an endogenous gene echR comprising the nucleic acid sequence SEQ ID. NO: 5.

A twenty-eighth embodiment, a strain according to one of the twentieth through the twenty-seventh embodiments, wherein the non-naturally occurring strain of Amycolaptosis sp., is a recombinant strain.

A twenty-ninth embodiment, a process for producing vanillin, comprising culturing non-naturally occurring strain according to any one of the twentieth through the twenty-eighth embodiments, including the steps of:
a. culturing said non-naturally occurring strain of Amycolaptosis sp., in an appropriate medium comprising a substrate, wherein at least a portion of the substrate is converted to vanillin by the activity of said non-naturally occurring strain of Amycolaptosis sp., and
b. recovering the produced vanillin.

A thirtieth embodiment, a method for producing vanillin comprising: culturing non-naturally occurring strain of Amycolaptosis sp., according to any one of the twentieth through the twenty-eighth embodiments, in a medium including a carbon source; and feeding ferulic acid to the mutant strain for enough time to allow conversion of ferulic acid to vanillin.

Other features and advantages of the present invention will become apparent in the following detailed description, taken with reference to the accompanying drawings.

### BRIEF DESCRIPTION OF THE SEQUENCES

Table 1 briefly describes the sequences disclosed herein and in the attached sequence listing. As known to the skilled artisan, it is noted that prokaryotes use alternate start codons, mainly GUG and UUG, which are translated as formyl-methionine.

TABLE 1
Sequence Information

| | |
|---|---|
| SEQ ID NO :. 1 | DNA; Nucleic Acid Sequence of gltB |
| SEQ ID NO: 2 | PRT; Amino Acid Sequence of GltB |
| SEQ ID NO: 3 | DNA; Nucleic Acid Sequence of gltD |
| SEQ ID NO: 4 | PRT; Amino Acid Sequence of GltD |
| SEQ ID NO: 5 | DNA; Nucleic acid sequence of echR |
| | (repressor of ech-fcs operon) |
| SEQ ID NO: 6 | PRT; Amino Acid Sequence of EchR |
| | (repressor of ech-fcs operon) |

### BRIEF DESCRIPTION OF THE DRAWINGS

For a better understanding of the present disclosure, reference may be made to the accompanying drawings.

FIG. 1. Metabolic pathway for vanillin within Amycolatopsis sp.

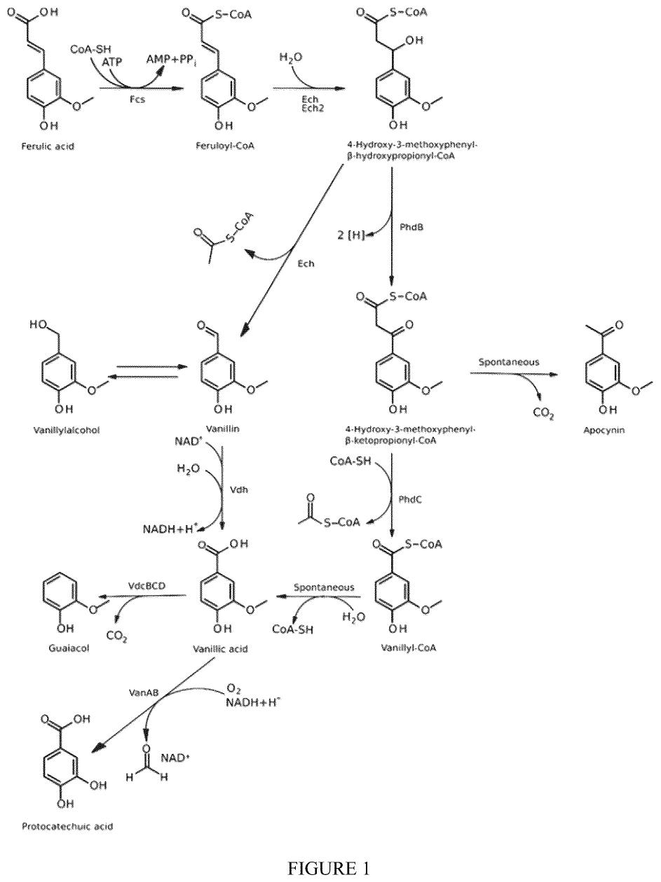

FIG. 2. Levels of vanillin, vanillic acid and vanillyl alcohol in wild type and mutant strains of Amycolatopsis sp. at the end of 44 hours of bioconversion process using ferulic acid as a feedstock. The two mutant strains 6-E11 and 12-H11 have mutations in the gltBD operon and a defective vanillic acid utilization pathway.

FIG. 3. Kinetics of ferulic acid utilization and production of vanillin, vanillic acid and vanillyl alcohol in the wild-type and 6-E11 strain of Amycolatopsis sp. The mutant strains 6-E11 has mutations in the gltBD operon and a defective vanillic acid utilization pathway.

### DETAILED DESCRIPTION

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of ectopic proteins. It included bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes for a specific amino acid sequence.

“Growing” or “cultivating” a cellular system includes providing an appropriate medium that would allow cells to multiply and divide. It also includes providing resources so that cells or cellular components can translate and make recombinant proteins.

The term “complementary” is to be given its ordinary and customary meaning to a person of ordinary skill in the art and is used without limitation to describe the relationship between nucleotide bases that are capable of hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing vanillin.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In one aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosome. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

As defined in the present invention, an organism comprising an exogenous nucleic acid derived from another organism is considered as a genetically modified organism (GMO). If an organism is genetically modified without the introduction of an exogenous nucleic acid molecule, it could be considered as non-genetically modified organism (non-GMO). A non-GMO may have one or more genetic modifications in its endogenous nucleic acid such as a point mutation in the coding sequence of a gene or deletion of an entire coding region of a gene without having any exogenous nucleic acid sequence.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention, “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “Best Fit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981; Smith et al., NUCLEIC ACIDS RESEARCH 11:2205-2220, 1983). The percent identity is most preferably determined using the “Best Fit” program

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% sequence identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% sequence identity, at least about 90% sequence identity, or even greater sequence identity, such as about 98% or about 99% sequence identity with a polynucleotide sequence described herein. Polynucleotide molecules that have the activity genes of the current invention are capable of directing the production of vanillin and have a substantial percent sequence identity to the polynucleotide sequences provided herein and are encompassed within the scope of this invention.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following: BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR). In molecular cloning, a vector is a DNA molecule used as a vehicle to artificially carry foreign genetic material into another cell, where it can be replicated and/or expressed (e.g. plasmid, cosmid, Lambda phages). A vector containing foreign DNA is considered recombinant DNA. The four major types of vectors are plasmids, viral vectors, cosmids, and artificial chromosomes. Of these, the most commonly used vectors are plasmids. Common to all engineered vectors are an origin of replication, a multicloning site, and a selectable marker.

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provided are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR-amplified polynucleotide can then be cloned into the LIC vector without restriction digest or ligation (Aslanidis and de Jong, NUCL. ACID RES. 18 6069-74, (1990); Haun et al., BIOTECHNIQUES 13, 515-18 (1992), each of which are incorporated herein by reference).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, and place the coding region in the desired reading frame.

In an embodiment, a polynucleotide for incorporation into an expression vector of the subject technology is prepared using PCR appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

### Fermentative Production of Vanillin

Ferulic acid is metabolized within Amycolatopsis sp. into vanillin through a non-oxidative deacetylation pathway mediated by two enzymes, namely, feruloyl-coenzyme A (CoA) synthetase coded by fcs gene and enoyl-CoA hydratase/aldolase coded by ech gene. Both genes (ech and fcs) are within a single operon. Wild-type and mutant strains of Amycolatopsis sp. have been used in the bioconversion process for producing vanillin using ferulic acid as feedstock.

One major challenge associated with the use of Amycolatopsis sp. ATCC 39116 for the bioconversion of ferulic acid to vanillin is the concurrent of degradation of vanillin to vanillic acid by vanillin dehydrogenase Vdh coded by the vdh gene. As a result of the action of vanillin dehydrogenase on vanillin, the yield for vanillin in the bioconversion process using ferulic acid as the feedstock is significantly reduced. Efforts have been made to functionally inactivate vanillin dehydrogenase through genetic manipulation of the vdh gene. The present invention provides novel mutant strains of Amycolatopsis sp. exhibiting a significant decrease in the degradation of vanillin to vanillic acid without directly deleting or inactivating the vdh gene. The mutant strains of Amycolatopsis sp. of the present invention was obtained using chemical mutagenesis and the nature of the genetic mutations conferring the phenotype of reduced vanillin degradation to vanillic acid was identified by using whole genome sequencing.

The present invention provides a protocol involving mutagenic chemicals for increasing the mutagenic frequency in gene(s) coding for proteins (e.g., enzymes and regulatory proteins) that participate in the vanillic acid utilization pathway.

A number of different chemical mutagenic agents are well known in the art. Any one of them can be used in the present invention to increase the frequency of mutations in one or more genes coding for proteins (e.g., enzymes and/or regulatory proteins) that participate in the vanillic acid utilization pathway. One class of chemical mutagenic agents includes base analogues which are similar to one of the four bases of DNA. Incorporation of such a base analogue would cause a stable mutation. Nitrous oxide, another mutagenic chemical, can convert the amino group of bases into keto group through oxidative deamination. The order of frequency of deamination (removal of amino group) is adenine>cytosine>guanine. Alkylating agents is another group of mutagenic agents useful in adding an alkyl group to the hydrogen bonding oxygen of guanine and adenine residues of DNA. As a result of alkylation, the possibility of ionization is increased with the introduction of pairing errors. Some widely used examples of alkylating agents include dimethyl sulphate, ethyl methane sulphonate, ethyl ethane sulphonate and methyl methane sulphonate. Certain dyes such as acridine orange, proflavine and acriflavin which are three ringed molecules of similar dimensions as those of purine pyrimidine pairs also can be used. In aqueous solutions, these dyes can insert themselves in DNA between the bases in adjacent pairs by a process called intercalation. These intercalating agents distort the DNA, resulting in deletion or insertion after replication of the DNA molecule. Due to such deletion or insertion caused by the intercalating agents, frameshift mutations can occur. Any of the aforementioned categories of mutagenic agents can be used in the present invention.

In one aspect, mutant strains with the desired phenotype (in this case, reduced vanillin degradation) can be obtained by subjecting spores of a vanillin-producing organism such as Amycolatopsis sp. to chemical mutagens following procedures well known in the art. Mutants having genetic mutations that affect the vanillin degradation pathway are selected. Specifically, the selected mutants show a defective vanillic acid utilization pathway. Because degradation of vanillin creates a source of energy for Amycolatopsis sp. cells, screening for mutants that can no longer grow when vanillin is the sole carbon source would uncover mutations that affect the vanillin degradation pathway. In addition, because vanillyl alcohol is converted to vanillin by Amycolatopsis sp. cells and vanillyl alcohol is less toxic to Amycolatopsis sp. cells than vanillin, the screening also can be performed by using vanillyl alcohol instead of vanillin as the sole carbon source. In addition, the present screening method also comprises adding an antibiotic such as penicillin to the liquid medium (what is known as the penicillin enrichment technique). The penicillin enrichment technique takes advantage of the fact that penicillin can kill only growing cells by inhibiting the cross-linking of peptidoglycan polymers essential for the structural integrity of the cell wall. Because Amycolatopsis sp. cells with a mutation in the vanillic acid utilization pathway will not be able to grow and undergo cell division in a growth medium containing vanillin or vanillyl alcohol as the sole source of energy, they will survive the penicillin treatment. Meanwhile, wild-type Amycolatopsis sp. cells that are growing and feeding on vanillin or vanillyl alcohol are killed by the penicillin. Surviving cells following the penicillin treatment are plated, and samples from individual colonies are taken and screened using ferulic acid bioconversion assay to identify mutant strains with the desired phenotype; namely, a relative increase in the ratio of vanillin to vanillic acid when compared to the wild-type strain. In preferred embodiments, the surviving cells are plated in the same medium containing penicillin and either vanillin or vanillyl alcohol, to avoid auxotrophic mutants. sp.

Once a mutant strain with a defective vanillic acid utilization pathway is selected, the nature of the mutations causing the observed phenotype is identified using whole genome sequencing. In the present invention, mutations in the gltBD operon were found to be associated with the observed phenotype. More specifically, a mutation in the gltB gene comprising nucleic acid sequence of SEQ ID NO: 1 and/or a mutation in the gltD gene comprising nucleic acid sequence of SEQ ID NO: 3 were found to be associated with the observed phenotype of reduced degradation of vanillin to vanillic acid. The one or more mutations in the gltB gene and/or the gltD gene can be selected from the group consisting of a deletion, an insertion, a frameshift mutation, a promoter mutation, a missense mutation, a nonsense mutation, a slicing mutation, a point mutation, and combinations thereof, where the mutation is capable of causing functional inactivation of the vanillic acid utilization pathway, leading to reduced degradation of vanillin to vanillic acid.

The role of the identified mutation(s) in inducing the observed phenotype can be verified by means of introducing the identified mutation(s) into wild-type Amycolatopsis sp. cells and demonstrating that the introduced mutation(s) do confer the desired phenotype. The introduction of the identified mutation(s) into wild-type Amycolatopsis sps cells can be carried out using one or more techniques well known in the field of microbial genetics. In a preferred aspect of the present invention, the introduction of the identified mutation in the gltBD operon into wild-type Amycolatopsis sp. cells is carried out using a technique so that no exogenous nucleic acid is introduced into the Amycolatopsis sp. cells for the purpose of maintaining the status of non-GMO.

Once a mutant strain of Amycolatopsis sp. is selected for reduced degradation of vanillin to vanillic acid, the performance of this strain can further be improved, by means of improving the bioconversion efficiency for vanillin production using ferulic acid as a substrate. The bioconversion of ferulic acid to vanillin in the wild-type Amycolatopsis sp. strain typically exhibits a lag period for 4-5 hours. This lag period is due to the repression of the expression of the feruloyl-coenzyme A (CoA) synthetase coded by the fcs gene and the enoyl-CoA hydratase aldolase coded by the ech gene. The repressed expression of these two genes is regulated by the repressor protein EchR (SEQ ID NO: 6) coded by the echR gene (SEQ ID NO: 5). Repression of the expression of the ech and fcs genes can be overcome by introducing an appropriate mutation in the echR gene leading to a functional inactivation of the EchR repressor protein. Using techniques well known in the art, one can introduce one or more mutations, including but not limited to a deletion, an insertion, a frameshift mutation, a promoter mutation, a missense mutation, a nonsense mutation, a slicing mutation, a point mutation, or any combination thereof, which can be used to functionally inactivate the EchR repressor protein. In a preferred embodiment, the mutation of the echR gene leading to the functional inactivation of EchR repressor protein is carried out using one or more techniques well known in the art of microbial genetics which would cause the desired mutation in the echR gene without introducing any exogeneous nucleic acid sequence into the selected mutant strain of Amycolatopsis sp. so that the status of non-GMO is maintained.

It has been reported that enzymes involved in the conversion of benzaldehyde to benzyl alcohol in Escherichia coli are also responsible for the reduction of vanillin to vanillyl alcohol. Deletion of yeaE, dkgA, yqhC, yqhD, yahK and yjgB from E. coli has eliminated the further reduction of vanillin. One can identify the homologs for these genes in the mutant strains Amycolatopsis sp. The inactivation of one or more of those genes would result in the reduction or elimination of vanillyl alcohol production resulting in an increase in vanillin yield.

In a preferred embodiment of the present invention, any further genetic modification to the mutant Amycolatopsis sp. cells of the present invention can be done using the CRISPAR/CAS system well known in the field of microbial genetics (see, e.g., US Patent Application Publication 2016/0298096—CRISPR-CAS System, Materials and Methods; Wang et al. (2016), Bacterial Genome Editing with CRISPR-Cas9: Deletion, Integration, Single Nucleotide Modification, and Desirable “Clean” Mutant Selection in Clostridium beijerinckii as an Example, ACS Synth. Biol., DOI: 10.1021/acssynbio.6b00060; Huang et al. (2016), CRISPR interference (CRISPRi) for gene regulation and succinate production in cyanobacterium S. elongatus PCC 7942, Microb Cell Fact, 15:196; Kuivanen et al. (2016), Engineering Aspergillus niger for galactaric acid production: elimination of galactaric acid catabolism by using RNA sequencing and CRISPR/Cas9, Microb Cell Fact, 15:210; Peng et al. (2017), Efficient gene editing in Corynebacterium glutamicum using the CRISPR/Cas9 system, Microb Cell Fact, 16:201; Gorter de Vries et al. (2017), CRISPR-Cas9 mediated gene deletions in lager yeast Saccharomyces pastorianus, Microb Cell Fact, 16:222; Wu et al. (2019), Strategies for Developing CRISPR-Based Gene Editing Methods in Bacteria, Small Methods, DOI: 10.1002/smtd.201900560; Ramachandran G and Bikard D (2019), Editing the microbiome the CRISPR way, Phil. Trans. R. Soc. B, 374: 20180103 http://dx.doi.org/10.1098/rstb.2018.0103). sp.

In some embodiments of the present invention, the functional enzymes can be inactivated using antisense RNA technology or RNAi technology (see, e.g., Xu et al. (2018), Antisense RNA: the new favorite in genetic research, Biomed & Biotechnol., 19(10):739-749; Zheng et al. (2019), Microbial CRISPRi and CRISPRa Systems for Metabolic Engineering, Biotechnology and Bioprocess Engineering, 24: 579-591. However, in the spirit of the present invention for using non-GMO strains in the bioconversion of ferulic acid to vanillin, it is necessary to make sure that the use of antisense RNA technology and RNAi technology to inactivate a functional protein does not introduce any exogenous nucleic acid into the mutant Amycolatopsis sp. strains selected for the production of vanillin from ferulic acid in a commercial scale and the non-GMO status is maintained.

The culture broth can be prepared and sterilized in a bioreactor. Engineered host strains according to the present invention can then be inoculated into the culture broth to initiate the growth phase. An appropriate duration of the growth phase can be about 5-40 hours, preferably about 10-35 hours and most preferably about 10-20 hours.

After the termination of the growth phase, the substrate ferulic acid can be fed to the culture. A suitable amount of substrate-feed can be 0.1-40 g/L of the fermentation broth, preferably about 0.3-30 g/L. The substrate can be fed as a solid material or as an aqueous solution or suspension. The total amount of substrate can be either fed in one step, in two or more feeding-steps, or continuously.

The bioconversion phase using the Amycolatopsis sp. strains developed in the present invention starts with the beginning of the substrate feed and can last about 5-50 hours, preferably 10-40 hours, and most preferably 15-30 hours, until all substrate is converted to product and by-products. Unlike the wild type Amycolatopsis sp. cells showing significant degradation of vanillin to vanillic acid, the Amycolatopsis sp. strains developed in the present invention shows little accumulation of vanillic acid throughout the vanillin production process. For example, a mutant strain of Amycolaptosis sp. according to the present invention can show accumulation of less than 0.5 g of vanillic acid per liter of growth medium at more than 24 hours after ferulic acid is initially fed to the mutant strain. In another embodiment, a mutant strain of Amycolaptosis sp. according to the present invention can show accumulation of less than 0.25 g of vanillic acid per liter of growth medium at more than 24 hours after ferulic acid is initially fed to the mutant strain. In yet another embodiment, a mutant strain of Amycolaptosis sp. according to the present invention can show accumulation of less than 0.5 g of vanillic acid per liter of growth medium at more than 44 hours after ferulic acid is initially fed to the mutant strain. In another embodiment, the present mutant strain can show accumulation of less than 0.25 g of vanillic acid per liter of growth medium at more than 44 hours after ferulic acid is initially fed to the mutant strain. In embodiments where the mutant strain further comprises a mutation in the echR gene, the mutant strain also will be able to produce vanillin right away with the introduction of ferulic acid without showing any lag period.

After the terminated bioconversion phase, the biomass can be separated from the fermentation broth by any well-known methods, such as centrifugation or membrane filtration and the like to obtain a cell-free fermentation broth.

An extractive phase can be added to the fermentation broth using, e.g., a water-immiscible—organic solvent, a plant oil or any solid extractant, e.g., a resin; preferably, a neutral resin. The fermentation broth can be further sterilized or pasteurized. In some embodiments, the fermentation broth can be concentrated. From the fermentation broth, vanillin can be extracted selectively using, for example, a continuous liquid-liquid extraction process, or a batch-wise extraction process.

Advantages of the present invention include, among others, the ability of the Amycolatopsis sp. strains developed in the present invention to start the bioconversion of ferulic acid to vanillin without any lag period to shorten the production period. This highly simplifies the production process, making the process efficient and economical, thus allowing scale-up to industrial production levels.

One skilled in the art will recognize that the vanillin composition produced by the method described herein can be further purified and mixed with fragrant and/or flavored consumable products as described above, as well as with dietary supplements, medical compositions, and cosmeceuticals, for nutrition, as well as in pharmaceutical products.

The disclosure will be more fully understood upon consideration of the following non-limiting examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### Examples

### Bacterial Strains, Plasmids and Culture Conditions.

E. coli strains of DH5a and BL21 (DE3) were purchased from Invitrogen. Plasmid pET28a was purchased from EMD Millipore (Billerica, MA, USA), which was used for gene cloning.

### DNA Manipulation.

All DNA manipulations were performed according to standard procedures. Restriction enzymes, T4 DNA ligase were purchased from New England Biolabs. All PCR reactions were performed with New England Biolabs' Phusion PCR system according to the manufacturer's guidance.

### Example 1

### Chemical Mutagenesis and Screening for Mutant Strains

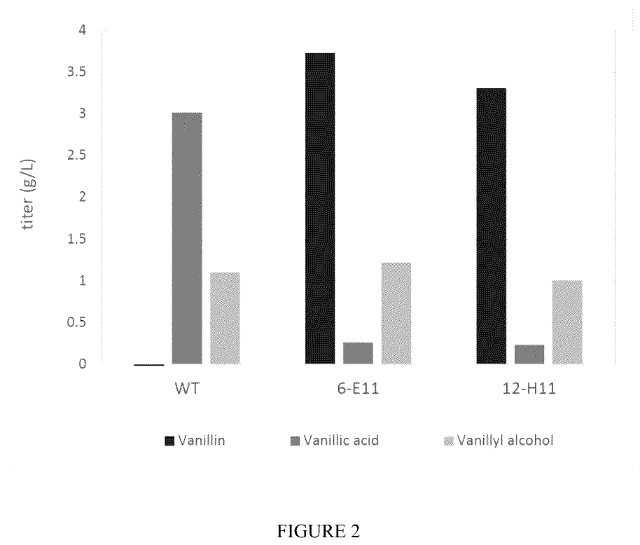

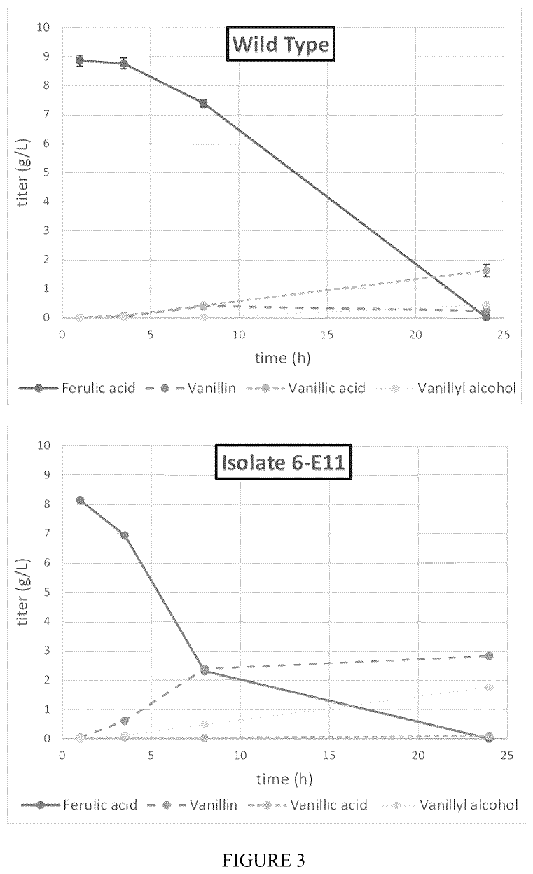

Single-celled spores of Amycolatopsis sp. ATCC 39116 were treated with the mutagen methyl methanesulfonate and then grown in liquid medium with vanillyl alcohol as the sole source of energy and 300 micrograms/milliliter (μg/ml) penicillin for 24 hours and plated on the same medium to avoid auxotrophic mutants. Single colonies were picked and tested in a ferulic acid bioconversion assay, using HPLC to assess vanillin levels. Out of 1414 colonies tested, two mutant strains (6-E11 and 12-H11) had the desired phenotype of reduced degradation of vanillin (FIG. 2). Further analysis with the mutant strain 6-E11 established the kinetic profile for the production vanillin, vanillic acid and vanillyl alcohol. The vanillin production with the mutant strain 6-E11 did not show any lag period as observed in the wild-type strain of Amycolatopsis sp. As shown in FIG. 3, the mutant strain 6-E11 began the conversion of ferulic acid to vanillin approximately 4-5 h before wild-type strain of Amycolatopsis sp. Compared to previously reported vdh mutant strains which did not exhibit vanillin production without any lag period, the mutant stains according to the present invention therefore confer both the advantages of reduced degradation of vanillin to vanillic acid as well as vanillin production without a lag period.

### Example 4

### Identification of Mutant Gene

Whole genome sequencing was performed with the two selected mutant strains to identify the mutation(s) causing the observed phenotype of reduced degradation of vanillin to vanillic acid. Whole-genome sequencing revealed that the two mutant strains have independent mutations, but both are in genes in the gltBD operon coding for the NADPH-dependent glutamate synthase enzyme complex (locus tags AMY39116_RS0321350 and AMY39116_RS0321345). These mutations are in two subunits of the NADPH-dependent glutamate synthase enzyme complex, which catalyzes the reaction: L-glutamine+2-oxoglutarate+NADPH+H⁺→L-glutamate+NADP⁺. Mutant strain 6-E11 contains a 2 bp insertion in the gltD gene after bp 1161, creating a frameshift and truncating the protein after 45 additional amino acid residues. Mutant strain 12-H11 contains a 2 bp insertion after bp 3148 in the gltB gene, creating a frameshift and truncating the protein after seven additional amino acids.

### Example 5

### HPLC Analysis

The HPLC analysis of vanillin was carried out with a Vanquish Ultimate 3000 system. The compounds were identified by their retention times, as well as the corresponding spectra, which were identified with a diode array detector in the system.

### Example 6

### Bioconversion of Ferulic Acid to Vanillin

Wild-type and mutant strains of Amycolatopsis sp. were grown to saturation in 10 mL seed medium (yeast extract 12 g/L, glucose 10 g/L, MgSO₄ 0.2 g/L, K₂HPO₄ 7.5 g/L, KH₂PO₄ 1 g/L, pH 7.2) for 24 hours at 37° C., diluted 1:20 into 10 mL conversion medium (yeast extract 5 g/L, glucose 8 g/L, malt extract 10 g/L, MgSO₄ 0.2 g/L) for 24 hours at 37° C. and fed with ferulic acid as a substrate. Samples were taken at the indicated times by collection of the bacterial cultures into methanol for HPLC analysis.

Referring to FIG. 2, it can be seen that even after 44 hours of conversion time, the mutant strains 6-E11 and 12-H11 according to the present invention showed very little degradation of vanillin compared to the wild-type (WT) strain which did not retain any vanillin. After 44 hours of conversion time, it appears that all the vanillin in the wild-type strain has been degraded to vanillic acid. More specifically, the accumulation of vanillic acid in the wild-type strain was approximately 3 g/L after 44 hours of conversion time. By comparison, mutant strain 6-E11 retained >3.5 g/L of vanillin and accumulated <0.25 g/L of vanillic acid. Similarly, mutant strain 12-H11 retained >3.0 g/L of vanillin and accumulated <0.25 g/L of vanillic acid.

Referring to FIG. 3, it can be seen that after 24 hours of conversion of 13 g/L ferulic acid in 2 mL of cells, the mutant strain 6-E11 produced much more vanillin than the WT strain (2.93 g/L versus 0 g/L) and less vanillic acid (0.130 g/L versus 1.16 g/L). In addition, the mutant strain 6-E11 also began vanillin production 4-5 hours earlier than the WT strain.

This application refers to various issued patents, published patent applications, journal articles, and other publications, all of which are incorporated herein by reference. All references, patents and patent applications disclosed herein are incorporated by reference with respect to the subject matter for which each is cited, which in some cases may encompass the entirety of the document. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above description, but rather is as set forth in the appended claims. Those of ordinary skill in the art will appreciate that various changes and modifications to this description may be made without departing from the spirit or scope of the present disclosure, as defined in the following claims.

Sequences of Interest

SEQ ID NO: 1: DNA; Nucleic Acid Sequence of gltB
ATGATCTTCTCCGCCAACCCGGGCAAGCAGGGCCTGTACGACCCTGCCATGGAGCAGGATTCCT
GCGGTGTGGCGATGGTGGCCGACATTCAGGGCCGGCGCACGCACGCCATCGTCACGGACGGGCT
GACGGCGCTGATCAACCTGGACCACCGGGGCGCCGCGGGCGCCGAACCGACTTCCGGCGACGGC
GCCGGGATCCTCGTGCAGCTGCCCGACCAGCTGCTCCGCGAGGAAGCCGGCTTCGAGCTGCCCG
AACCCGACGCGCAGGGCCACCACCGCTACGCGGCCGGCATCGCGTTCCTGCCCGCCGAGGAGGA
GGCGCGCGGCAAGGCCGTGGCGCTGATCGAACGCCTCGCCGACGAGGAGAGCCTCGAGGTGCTG
GGCTGGCGCGAGGTCCCGGTCGACGCCGACGGGGCGGACATCGGCCCCACCGCGCGTTCGGTGA
TGCCGCACTTCGCCATGCTGTTCGTGGCGGGCAGGCCGGACGCCGAGGGCGTGCGGCCCTCCGG
CCTCGCGCTGGACCGGCTCACCTTCTGCCTGCGCAAGCGCGTCGAGCACGAGAGCGTCGTGGCC
GAGTGCGGCACGTACTTCCCGTCGCTGTCCTCGCGCACCTTGGTCTACAAGGGAATGCTCACGC
CCGAGCAGCTCCCCGCGTTCTTCGGCGACCTGCGCGACCCGCGGCTCACCAGCGCGATCGCACT
GGTGCACAGCCGCTTTTCCACCAACACGTTCCCGTCGTGGCCGCTGGCGCACCCGTTCCGGTTC
GTCGCGCACAACGGTGAGATCAACACGATCCGCGGCAACCGCAACCGCATGCGGGCCCGCGAGG
CGCTGCTCGAATCGGGCCTGATCCCGGGCGACCTGACCCGGCTGTACCCGATCTGCTCGCCGGA
GGCGTCCGACTCGGCGTCCTTCGACGAGGTGCTCGAACTGCTGCACCTGGGCGGTCGCAGCCTG
CCGCACGCGGTGCTGATGATGATCCCGGAGGCGTGGGAGAACCACTCGACCATGGACGCGCAGC
GCCGCGCGTTCTACCAGTTCCACGCCAGCCTGATGGAGCCGTGGGACGGCCCCGCGTGCGTCAC
CTTCACCGACGGCACGCTCGTCGGCGCGGTGCTGGACCGCAACGGCCTGCGCCCGTGCCGCTGG
TGGCGCACCGCCGACGACCGCGTCGTGCTGGCCAGCGAGGCCGGCGTCCTGGACGTGCCGCCGG
ACCAGGTGGTCGCCAAGGGCCGCCTCAAGCCGGGCCGCATGTTCCTGGTGGACACCGAGGCAGG
CCGCATCGTCGCCGACGACGAGGTCAAGTCGGAGCTGGCGAAGCAGCACCCGTACGAGGAGTGG
CTGCACGCCGGCCTGCTGCAGCTGGCCGACCTGCAGGACCGCGACCACGTCACGCAGAGCCACG
ACTCGGTGCTGCGCCGCCAGCTCGCCTTCGGCTACTCCGAGGAGGAGCTGAAGATCCTGCTCGC
GCCGATGGCCGAGAAGGGCGCCGAGCCGCTGGGCTCGATGGGCACCGACACCCCGCCCGCCGTG
CTGTCCAAGCGCTCGCGGCAGCTCTACGACTACTTCAAACAGGCCTTCGCCCAGGTGACCAACC
CGCCGCTGGACGCGATCCGCGAGGAGCTGGTCACCTCGATGAGCCGGATCATGGGTCCCGAGCG
CAACCTGCTCGACCCTGGCCCGGCCTCGTGCCGGCACATCCAGCTGCCGTACCCGGTCATCGAC
AACGACGAGCTGGCCAAGCTCATCCACATCAACGACGACGGTGACCTGCCCGGCTTCGCCTGCA
CCGTCCTGTCCGGACTGTTCGAAGTGGACGGCGGCGGCAAGGCGCTGGCGGAGGCGATCGAGCG
GGTGCGCCGCGAGGCGTCCGAGGCGATCGCGGCGGGCGCGCGCACGCTCGTGCTGTCCGACCGG
GACTCCGACCACAAGATGGCGCCGATCCCGTCGCTGCTGCTGGTTTCCGCGGTGCACCACCACC
TGGTGCGCACCAAGGAGCGGCTGCGCGTCGCGCTCGTCGTCGAGACCGGTGACGCCCGCGAGGT
GCACCACATCGCGCTGCTGCTCGGTTACGGCGCGGCCGCGGTGAACCCGTACCTGGCCTTCGAG
ACGATCGAGGACATGATCGCGCAGGGCGCGATCACCGGCATCGAGCCGCGCAAGGCCGTGCGCA
ACTACGTCAACGCGCTCGTCAAGGGCGTCCTGAAGATCATGTCCAAGATGGGCATCTCGACCGT
CGGCGCCTACACCGCGGCGCAGGTGTTCGAGTCCTTCGGGCTGTCGCAGGAACTGCTCGACGAG
TACTTCACCGGCACGGTGTCCAAGCTCGGCGGCGTCGGTCTCGACGTGCTCGCCGAGGAGGTCG
CCGTCCGGCACCGCCGGGCGTACCCGGACAACCCCACCGACCGGGTGCACCGCCGCCTGGACAG
CGGCGGCGAGTACGCCTACCGCCGCGAGGGCGAGCTGCACCTGTTCACCCCGGAGACCGTGTTC
CTGCTGCAGCACGCCAGCAAGACGCGCCGCGACGAGGTGTACCGCAAGTACACCGAAGAGGTGC
ACCGCCTGTCCCGCGAGGGCGGTGCGCTGCGCGGGTTGTTCAAGTTCCGCAAGGAAGGCCGCGC
CCCGGTGCCGATCGACGAGGTCGAGTCGGTCGAGTCGATCTGCAAGCGGTTCAACACCGGCGCG
ATGTCCTACGGTTCGATCTCGGCCGAGGCGCACCAGACGCTCGCGATCGCGATGAACCGCATCG
GCGGCCGCTCCAACACCGGTGAGGGCGGCGAGGACCCGGAGCGGCTCTACGACCCCGAGCGGCG
CAGCGCGATCAAGCAGGTCGCCAGCGGCTGGTTCGGCGTGACGAGCGAGTACCTGGTCAACGCC
GACGACATCCAGATCAAGATGGCGCAGGGCGCCAAGCCCGGCGAGGGCGGCCAGCTGCCGCCGA
ACAAGGTGTACCCGTGGATCGCGCGCACCCGGCACTCCACGCCGGGCGTCGGCCTCATCTCGCC
GCCGCCGCACCACGACATCTACTCGATCGAGGACCTGGCGCAGCTGATCCACGACCTGAAGAAC
GCCAACGAGCAGGCCCGCATCCACGTGAAGCTGGTCAGCTCGCTCGGCGTCGGCACGGTCGCGG
CCGGCGTGTCCAAGGCGCACGCGGACGTCGTGCTGATCTCCGGCCACGACGGCGGCACCGGCGC
CTCGCCGCTGAACTCGCTCAAGCACGCGGGCACGCCGTGGGAGATCGGCCTTGCCGAGACCCAG
CAGACCCTGATGCTGAACGGGCTGCGCGACCGCATCACCGTGCAGGTCGACGGCGCGATGAAGA
CCGGCCGCGACGTCGTGGTCGCCGCGCTGCTCGGCGCCGAGGAGTACGGCTTCGCGACCGCGCC
GCTGATCGTGGCCGGCTGCATCATGATGCGCGTCTGCCACCTCGACACCTGCCCCGTCGGTGTC
GCCACCCAGAGCCCGGAGCTGCGCAAGCGCTACACCGGGCAGGCCGAGCACGTGGTGAACTACT
TCCGGTTCGTCGCGCAGGAGGTCCGGGAACTGCTGGCGGAGCTGGGTTTCCGCACCCTGGACGA
GGCGATCGGCCGGGCCGACGTGCTGGACACCGACGACGCCGTCGACCACTGGAAGGCCAGCGGC
CTGGACCTGTCGCCGATCTTCCAGATGCCGACCGACACCCCGTACGGCGGCGCCCGGCGCAAGA
TCCGCGAGCAGGACCACGGCCTCGAGCACGCTCTGGACCGCACGCTGATCCAGTTGTCCGAGGC
GGCGCTGGAGGACGCGCACCCGGTCCGGCTGGAACTGCCGGTGCGCAACGTCAATCGCACCGTC
GGCACACTGCTGGGCTCGGAGATCACCCGCCGCTACGGCGGGGAGGGCCTGCCCGACGGCACGA
TCCACATCCGGCTCACCGGGTCGGCGGGGCAGTCGCTGGGCGCGTTCCTGCCGCGCGGCGTCAC
GCTGGAGATGGTGGGCGACGCCAACGACTACGTCGGCAAGGGCCTGTCCGGCGGCCGCATCATC
GTGCGGCCGCACCCGGACGCGACGTTCGCCGCTGAACGTCAGGTCATCGCCGGCAACACGCTGG
CCTACGGCGCCACCGCGGGGGAGATGTTCCTGCGCGGGCATGTGGGCGAGCGGTTCTGCGTACG
CAACTCGGGCGCCACCGTCGTCGCCGAGGGCGTGGGCGACCACGCCTTCGAATACATGACCGGT
GGCCGTGCCGTGGTGCTCGGTCCGACCGGCCGCAACCTCGCCGCGGGCATGTCCGGCGGTATCG
GCTACGTCCTCGACCTCGACCAGGGCAGCGTCAACCGCGAGATGGTCGAGCTGCTCCCGCTCGA
GCCCGAGGATCTGAACTGGTTGAAGGACATCGTGACCCGTCACCACGAACTCACCCGCTCGGCG
GTCGCCGCCTCGCTGCTCGGCGATTGGCCGCGCCGGTCGGCGAGCTTCACGAAGGTCATGCCGC
GCGACTACAAGCGCGTGCTGGAGGCGACCAAGGCCGCGAAGGCCGCGGGCCGCGACGTCGACGA
GGCGATCATGGAGGTGGCGTCTCGTGGCTGA
SEQ ID NO: 2: PRT; Amino Acid Sequence of GltB
MIFSANPGKQGLYDPAMEQDSCGVAMVADIQGRRTHAIVTDGLTALINLDHRGAAGAEPT
SGDGAGILVQLPDQLLREEAGFELPEPDAQGHHRYAAGIAFLPAEEEARGKAVALIERLA
DEESLEVLGWREVPVDADGADIGPTARSVMPHFAMLFVAGRPDAEGVRPSGLALDRLTFC
LRKRVEHESVVAECGTYFPSLSSRTLVYKGMLTPEQLPAFFGDLRDPRLTSAIALVHSRF
STNTFPSWPLAHPFRFVAHNGEINTIRGNRNRMRAREALLESGLIPGDLTRLYPICSPEA
SDSASFDEVLELLHLGGRSLPHAVLMMIPEAWENHSTMDAQRRAFYQFHASLMEPWDGPA
CVTFTDGTLVGAVLDRNGLRPCRWWRTADDRVVLASEAGVLDVPPDQVVAKGRLKPGRMF
LVDTEAGRIVADDEVKSELAKQHPYEEWLHAGLLQLADLQDRDHVTQSHDSVLRRQLAFG
YSEEELKILLAPMAEKGAEPLGSMGTDTPPAVLSKRSRQLYDYFKQAFAQVTNPPLDAIR
EELVTSMSRIMGPERNLLDPGPASCRHIQLPYPVIDNDELAKLIHINDDGDLPGFACTVL
SGLFEVDGGGKALAEAIERVRREASEAIAAGARTLVLSDRDSDHKMAPIPSLLLVSAVHH
HLVRTKERLRVALVVETGDAREVHHIALLLGYGAAAVNPYLAFETIEDMIAQGAITGIEP
RKAVRNYVNALVKGVLKIMSKMGISTVGAYTAAQVFESFGLSQELLDEYFTGTVSKLGGV
GLDVLAEEVAVRHRRAYPDNPTDRVHRRLDSGGEYAYRREGELHLFTPETVFLLQHASKT
RRDEVYRKYTEEVHRLSREGGALRGLFKFRKEGRAPVPIDEVESVESICKRFNTGAMSYG
SISAEAHQTLAIAMNRIGGRSNTGEGGEDPERLYDPERRSAIKQVASGWFGVTSEYLVNA
DDIQIKMAQGAKPGEGGQLPPNKVYPWIARTRHSTPGVGLISPPPHHDIYSIEDLAQLIH
DLKNANEQARIHVKLVSSLGVGTVAAGVSKAHADVVLISGHDGGTGASPLNSLKHAGTPW
EIGLAETQQTLMLNGLRDRITVQVDGAMKTGRDVVVAALLGAEEYGFATAPLIVAGCIMM
RVCHLDTCPVGVATQSPELRKRYTGQAEHVVNYFRFVAQEVRELLAELGFRTLDEAIGRA
DVLDTDDAVDHWKASGLDLSPIFQMPTDTPYGGARRKIREQDHGLEHALDRTLIQLSEAA
LEDAHPVRLELPVRNVNRTVGTLLGSEITRRYGGEGLPDGTIHIRLTGSAGQSLGAFLPR
GVTLEMVGDANDYVGKGLSGGRIIVRPHPDATFAAERQVIAGNTLAYGATAGEMFLRGHV
GERFCVRNSGATVVAEGVGDHAFEYMTGGRAVVLGPTGRNLAAGMSGGIGYVLDLDQGSV
NREMVELLPLEPEDLNWLKDIVTRHHELTRSAVAASLLGDWPRRSASFTKVMPRDYKRVL
EATKAAKAAGRDVDEAIMEVASRG
SEQ ID NO: 3: DNA; Nucleic Acid Sequence of gltD
GTGGCTGACCCCAAGGGCTTCCTGAAGTACGAGCGGGTCGAGCCGCCCAAGCGCCCCAAGGAGC
ACCGCGCCGAGGACTGGCGCGAGGTCTACGTCGACCTCGAACCGGCCGAGCGCGACCAGCAGGT
GCGCACCCAGGCCACCCGCTGCATGGACTGCGGCATCCCGTTCTGCCACTCGGCCGGTTCCGGC
TGCCCGCTCGGCAACCTGATCCCGGAGTGGAACGACCTGGTGCGCCGCGGTGACTGGACCGCGG
CCAGCGACCGGCTGCACGCCACCAACAACTTCCCGGAGTTCACCGGGAAGCTGTGCCCGGCGCC
GTGCGAGGCGGGCTGCACGCTGTCCATCTCGCCGCTGTCCGGCGGCCCGGTCGCGATCAAGCGC
GTCGAGGCGACGATCGCGGAGAAGTCGTGGGAGCTGGGCCTGGCCCAGCCGCAGGTCGCCGAGG
TGGCCAGCGGTCAGCGCGTCGCCGTGGTCGGGTCCGGCCCGGCCGGTCTCGCCGCCGCCCAGCA
GCTCACCCGCGCCGGGCACGACGTGACCGTCTTCGAGCGGGACGACCGGCTCGGCGGGCTGCTC
CGATACGGCATCCCCGAGTTCAAGATGGAGAAGAAGCACCTCGACAAGCGCCTGGCCCAGCTCA
AGAAGGAGGGCACGCAGTTCGTCACGGGCTGCGAGGTGGGCGTCGACATCACCGTCGAGGAGCT
GCGGGCCCGCTACGACGCGGTCGTGCTCGCCGTCGGCGCGCTGCGCGGCCGCGACGACACCACC
ACGCCCGGCCGGGAGCTCAAGGGCATCCACCTGGCGATGGAGCACCTGGTGCCGGCCAACAAGC
AGTGCGAGGGCGACGGCCCGTCGCCGGTCCACGCGCACGGCAAGCACGTGGTGATCATCGGCGG
TGGTGACACCGGCGCCGACTCCTACGGCACCGCGATCCGCCAGGGCGCGGCCTCGGTGGTCCAG
CTGGACCAGTACCCGATGCCGCCGACGACCCGCGACGACGAGCGGTCGCCGTGGCCGACCTGGC
CGTACGTGCTGCGCACCTACCCGGCGCACGAGGAGGGCGGCGAGCGGAAGTTCGGTGTCGCCGT
GCGGCGGTTCGTGGGCGACGAGAACGGGCACGTCCGCGCGATCGAGCTGCAGCAGGTCAAGGTC
GTCAAGGACCCGGAGACCGGGCGCCGCGAGGTGCTGCCGGTGTCGGACGAGATCGAGGAGATCC
CGGCCGACCTGGTGCTCTTCGCCATCGGGTTCGAGGGCGTGGAGCACATGCGGCTGCTCGACGA
CCTGGGCATCCGGCTGACCCGGCGCGGCACCATCTCGTGCGGCCCGGACTGGCAGACCGAGGCC
CCGGGCGTGTTCGTCTGCGGTGACGCCCACCGCGGCGCGTCGCTGGTCGTGTGGGCGATCGCGG
AGGGCCGCTCGGTGGCCAACGCCGTCGACGCCTACCTGACCGGCGCGTCGGACCTGCCGGCCCC
GGTGCATCCGACGGCTCTGCCGCTCGCTGTGGTGTAA
SEQ ID NO: 4: PRT; Amino Acid Sequence of GltD
MADPKGFLKYERVEPPKRPKEHRAEDWREVYVDLEPAERDQQVRTQATRCMDCGIPFCHSAGSG
CPLGNLIPEWNDLVRRGDWTAASDRLHATNNFPEFTGKLCPAPCEAGCTLSISPLSGGPVAIKR
VEATIAEKSWELGLAQPQVAEVASGQRVAVVGSGPAGLAAAQQLTRAGHDVTVFERDDRLGGLL
RYGIPEFKMEKKHLDKRLAQLKKEGTQFVTGCEVGVDITVEELRARYDAVVLAVGALRGRDDTT
TPGRELKGIHLAMEHLVPANKQCEGDGPSPVHAHGKHVVIIGGGDTGADSYGTAIRQGAASVVQ
LDQYPMPPTTRDDERSPWPTWPYVLRTYPAHEEGGERKFGVAVRRFVGDENGHVRAIELQQVKV
VKDPETGRREVLPVSDEIEEIPADLVLFAIGFEGVEHMRLLDDLGIRLTRRGTISCGPDWQTEA
PGVFVCGDAHRGASLVVWAIAEGRSVANAVDAYLTGASDLPAPVHPTALPLAVV
SEQ ID NO: 5: DNA; Nucleic acid sequence of echR (repressor of ech-
fcs operon)
GTG GTG ACC GAA TCC CGC GCC GAG GAC GCC CCG CTG ACC CTC TAC CTG
GTC AAG CGG CTG GAG CTG GTG ATC CGC TCG CTG ATG GAC GAC GCG CTG
CGC CCG TTC GGG CTG ACC ACC CTG CAG TAC ACC GCG CTG ACC GCG CTG
CGG CAC CGC AAC GGG CTG TCG TCC GCG CAG CTC GCG CGC CGC TCG TTC
GTC CGG CCC CAG ACC ATG CAC ACC ATG GTG CTC ACG CTG GAG AAG TAC
GGG CTC ATC GAG CGC GCG GAG GAC CCG GCC AAC CGC CGG GTC CTG CTC
GCC ACC CTC ACC GAG CGC GGC AAG CAG GTC CTC GAC GAG TGC ACG CCG
CTG GTC CGG GAG CTC GAA GAC CGG ATG CTC TCC GGC ATG GAC GAC GAC
CGC CGC GCC GGG TTC CGC CGG GAC CTG GAG GAC GGC TAC GGC ATG CTC
GCC TCG CAC GCC AAC GCT CAG CGC GCG TTG ACG AAC GGC GGC GGC GAG
TAA
SEQ ID NO: 6: PRT; Amino Acid Sequence of EchR (repressor of ech-fcs
operon)
MVTESRAEDAPLTLYLVKRLELVIRSLMDDALRPFGLTTLQYTALTALRHRNGLSSAQLARRSF
VRPQTMHTMVLTLEKYGLIERAEDPANRRVLLATLTERGKQVLDECTPLVRELEDRMLSGMDDD
RRAGFRRDLEDGYGMLASHANAQRALTNGGGE

1.-30. (canceled)

31. A non-GMO mutant strain of Amycolaptosis sp., comprising: occurring mutant strain of Amycolaptosis sp., formed by exposing a strain of Amycolaptosis sp., capable of producing vanillin and of metabolizing vanillin to vanillic acid to at least one mutagen, wherein said mutant strain of Amycolaptosis sp., is capable of producing vanillin and exhibits less degradation of vanillin to vanillic acid than does the strain, as measured by the accumulation level of vanillic acid in said mutant strain versus in the strain.

32. The mutant strain according to c, wherein the mutant strain of Amycolaptosis sp., is a mutant of ATCC 39116.

33. The mutant strain of claim 31, wherein said mutant strain of Amycolaptosis sp., accumulates less than 0.5 grams of vanillic acid per liter of the culture medium at more than 24 hours after ferulic acid is initially fed to the mutant strain.

34. The mutant strain of claim 31, wherein said mutant strain of Amycolaptosis sp., accumulates less than 0.25 grams of vanillic acid per liter culture medium at more than 24 hours after ferulic acid is initially fed to the mutant strain.

35. The mutant strain of claim 31, wherein said mutant strain of Amycolaptosis sp., accumulates less than 0.5 grams of vanillic acid per liter culture medium at more than 44 hours after ferulic acid is initially fed to the mutant strain.

36. The mutant strain of claim 31, wherein said mutant strain of Amycolaptosis sp., accumulates less than 0.25 grams of vanillic acid per liter culture medium at more than 44 hours after ferulic acid is initially fed to the mutant strain.

37. The mutant strain of claim 31, wherein the genome of said mutant strain of Amycolaptosis sp., comprises one or more mutations in the gltBD operon.

38. The mutant strain of claim 37, wherein the one or more mutations in the gltBD operon includes at least one mutation in the gltB gene.

39. The mutant strain of claim 38, wherein the one or more mutations are in the gltB gene comprise a nucleic acid sequence having at least 90 percent identity to SEQ ID NO: 1.

40. The mutant strain of claim 38, wherein the one or more mutations are in the gltB gene comprising SEQ ID NO: 1.

41. The mutant strain of claim 37, wherein the one or more mutations in the gltBD operon are in the gltD gene comprising a nucleic acid sequence having at least 90 percent identity to SEQ ID NO: 3.

42. The mutant strain of claim 37, wherein the one or more mutations in the gltBD operon are in the gltD gene comprising nucleic acid sequence SEQ ID NO: 3.

43. The mutant strain of claim 37, further including a mutation in an endogenous gene echR comprising the nucleic acid sequence SEQ ID. NO 5.

44. The mutant strain of claim 37, wherein said one or more mutations are at least one mutation selected from the group consisting of a deletion, an insertion, a frameshift mutation, a missense mutation, a nonsense mutation, a slicing mutation, and a point mutation.

45. The mutant strain of claim 44, wherein said mutation is a frameshift mutation comprising a 2 bp insertion.

46. The mutant strain of claim 31, wherein the mutant strain is obtained without permanently modifying the mutant strain by introducing any exogenous genetic material.

47. The mutant strain of claim 31, wherein the mutant stain is obtained by contacting a strain with the mutagen methyl methanesulfonate.

48. A process for producing vanillin, comprising the steps of:
a. culturing the mutant strain according to claim 31 in an appropriate medium comprising a substrate; and
b. recovering the produced vanillin.

49. A method for producing vanillin comprising:
a. culturing a mutant strain of Amycolaptosis sp., according to claim 31 in a medium containing a carbon source; and
b. feeding ferulic acid to the mutant strain for enough time to allow conversion of ferulic acid to vanillin.

50. A strain of Amycolaptosis sp., comprising:
a. a non-naturally occurring strain of Amycolaptosis sp., that includes a gene encoding vanillin dehydrogenase and at least one mutation in the gltBD operon, wherein said strain of Amycolaptosis sp., converts less vanillin to vanillic acid than does a strain of Amycolaptosis sp., without said at last one mutation in gltBD operon, wherein the gltBD operon incudes a gltB gene and a gltD gene.

51. The strain according to claim 50, wherein the one or more mutations in the gltBD operon include at least one mutation in the gltB gene.

52. The strain according to claim 51, wherein the one or more mutations are in the gltB gene comprise a nucleic acid sequence having at least 90 percent identity to SEQ ID NO: 1.

53. The strain according to claim 51, wherein the one or more mutations are in the gltB gene comprising SEQ ID NO: 1.

54. The strain according to claim 50, wherein the one or more mutations in the gltBD operon are in the gltD gene.

55. The strain according to claim 54, wherein the one or more mutations in the gltD gene comprises a nucleic acid sequence having at least 90 percent identity to SEQ ID NO: 3.

56. The strain according to claim 54, wherein the one or more mutations in the gltD gene comprises nucleic acid sequence SEQ ID NO: 3.

57. The strain of claim 50, further including a mutation in an endogenous gene echR comprising the nucleic acid sequence SEQ ID. NO 5.

58. The strain of claim 50 wherein, the non-naturally occurring strain of Amycolaptosis sp., is a recombinant strain.

59. A process for producing vanillin, comprising culturing non-naturally occurring strain according to any one of claim 50 including the steps of:
a. culturing said non-naturally occurring strain of Amycolaptosis sp., in an appropriate medium comprising a substrate, wherein at least a portion of the substrate is converted to vanillin by the activity of said non-naturally occurring strain of Amycolaptosis sp.; and
b. recovering the produced vanillin.

60. A method for producing vanillin comprising:
a. culturing non-naturally occurring strain of Amycolaptosis sp., according to any one of claim 50 in a medium including a carbon source; and
b. feeding ferulic acid to the mutant strain for enough time to allow conversion of ferulic acid to vanillin.


SEQUENCE LISTING (6 sequences)

SEQ ID NO: 1 - DNA, 4575 bp, Amycolatopsis sp. ATCC 39116
  misc_feature (1)..(4575): Nucleic Acid Sequence of gltB
  misc_feature (4573)..(4575): stop codon
atgatcttctccgccaacccgggcaagcagggcctgtacgaccctgccatggagcaggattcctgcggtgtggcgatggtggccgacattcagggccggcgcacgcacgccatcgtcacggacgggctgacggcgctgatcaacctggaccaccggggcgccgcgggcgccgaaccgacttccggcgacggcgccgggatcctcgtgcagctgcccgaccagctgctccgcgaggaagccggcttcgagctgcccgaacccgacgcgcagggccaccaccgctacgcggccggcatcgcgttcctgcccgccgaggaggaggcgcgcggcaaggccgtggcgctgatcgaacgcctcgccgacgaggagagcctcgaggtgctgggctggcgcgaggtcccggtcgacgccgacggggcggacatcggccccaccgcgcgttcggtgatgccgcacttcgccatgctgttcgtggcgggcaggccggacgccgagggcgtgcggccctccggcctcgcgctggaccggctcaccttctgcctgcgcaagcgcgtcgagcacgagagcgtcgtggccgagtgcggcacgtacttcccgtcgctgtcctcgcgcaccttggtctacaagggaatgctcacgcccgagcagctccccgcgttcttcggcgacctgcgcgacccgcggctcaccagcgcgatcgcactggtgcacagccgcttttccaccaacacgttcccgtcgtggccgctggcgcacccgttccggttcgtcgcgcacaacggtgagatcaacacgatccgcggcaaccgcaaccgcatgcgggcccgcgaggcgctgctcgaatcgggcctgatcccgggcgacctgacccggctgtacccgatctgctcgccggaggcgtccgactcggcgtccttcgacgaggtgctcgaactgctgcacctgggcggtcgcagcctgccgcacgcggtgctgatgatgatcccggaggcgtgggagaaccactcgaccatggacgcgcagcgccgcgcgttctaccagttccacgccagcctgatggagccgtgggacggccccgcgtgcgtcaccttcaccgacggcacgctcgtcggcgcggtgctggaccgcaacggcctgcgcccgtgccgctggtggcgcaccgccgacgaccgcgtcgtgctggccagcgaggccggcgtcctggacgtgccgccggaccaggtggtcgccaagggccgcctcaagccgggccgcatgttcctggtggacaccgaggcaggccgcatcgtcgccgacgacgaggtcaagtcggagctggcgaagcagcacccgtacgaggagtggctgcacgccggcctgctgcagctggccgacctgcaggaccgcgaccacgtcacgcagagccacgactcggtgctgcgccgccagctcgccttcggctactccgaggaggagctgaagatcctgctcgcgccgatggccgagaagggcgccgagccgctgggctcgatgggcaccgacaccccgcccgccgtgctgtccaagcgctcgcggcagctctacgactacttcaaacaggccttcgcccaggtgaccaacccgccgctggacgcgatccgcgaggagctggtcacctcgatgagccggatcatgggtcccgagcgcaacctgctcgaccctggcccggcctcgtgccggcacatccagctgccgtacccggtcatcgacaacgacgagctggccaagctcatccacatcaacgacgacggtgacctgcccggcttcgcctgcaccgtcctgtccggactgttcgaagtggacggcggcggcaaggcgctggcggaggcgatcgagcgggtgcgccgcgaggcgtccgaggcgatcgcggcgggcgcgcgcacgctcgtgctgtccgaccgggactccgaccacaagatggcgccgatcccgtcgctgctgctggtttccgcggtgcaccaccacctggtgcgcaccaaggagcggctgcgcgtcgcgctcgtcgtcgagaccggtgacgcccgcgaggtgcaccacatcgcgctgctgctcggttacggcgcggccgcggtgaacccgtacctggccttcgagacgatcgaggacatgatcgcgcagggcgcgatcaccggcatcgagccgcgcaaggccgtgcgcaactacgtcaacgcgctcgtcaagggcgtcctgaagatcatgtccaagatgggcatctcgaccgtcggcgcctacaccgcggcgcaggtgttcgagtccttcgggctgtcgcaggaactgctcgacgagtacttcaccggcacggtgtccaagctcggcggcgtcggtctcgacgtgctcgccgaggaggtcgccgtccggcaccgccgggcgtacccggacaaccccaccgaccgggtgcaccgccgcctggacagcggcggcgagtacgcctaccgccgcgagggcgagctgcacctgttcaccccggagaccgtgttcctgctgcagcacgccagcaagacgcgccgcgacgaggtgtaccgcaagtacaccgaagaggtgcaccgcctgtcccgcgagggcggtgcgctgcgcgggttgttcaagttccgcaaggaaggccgcgccccggtgccgatcgacgaggtcgagtcggtcgagtcgatctgcaagcggttcaacaccggcgcgatgtcctacggttcgatctcggccgaggcgcaccagacgctcgcgatcgcgatgaaccgcatcggcggccgctccaacaccggtgagggcggcgaggacccggagcggctctacgaccccgagcggcgcagcgcgatcaagcaggtcgccagcggctggttcggcgtgacgagcgagtacctggtcaacgccgacgacatccagatcaagatggcgcagggcgccaagcccggcgagggcggccagctgccgccgaacaaggtgtacccgtggatcgcgcgcacccggcactccacgccgggcgtcggcctcatctcgccgccgccgcaccacgacatctactcgatcgaggacctggcgcagctgatccacgacctgaagaacgccaacgagcaggcccgcatccacgtgaagctggtcagctcgctcggcgtcggcacggtcgcggccggcgtgtccaaggcgcacgcggacgtcgtgctgatctccggccacgacggcggcaccggcgcctcgccgctgaactcgctcaagcacgcgggcacgccgtgggagatcggccttgccgagacccagcagaccctgatgctgaacgggctgcgcgaccgcatcaccgtgcaggtcgacggcgcgatgaagaccggccgcgacgtcgtggtcgccgcgctgctcggcgccgaggagtacggcttcgcgaccgcgccgctgatcgtggccggctgcatcatgatgcgcgtctgccacctcgacacctgccccgtcggtgtcgccacccagagcccggagctgcgcaagcgctacaccgggcaggccgagcacgtggtgaactacttccggttcgtcgcgcaggaggtccgggaactgctggcggagctgggtttccgcaccctggacgaggcgatcggccgggccgacgtgctggacaccgacgacgccgtcgaccactggaaggccagcggcctggacctgtcgccgatcttccagatgccgaccgacaccccgtacggcggcgcccggcgcaagatccgcgagcaggaccacggcctcgagcacgctctggaccgcacgctgatccagttgtccgaggcggcgctggaggacgcgcacccggtccggctggaactgccggtgcgcaacgtcaatcgcaccgtcggcacactgctgggctcggagatcacccgccgctacggcggggagggcctgcccgacggcacgatccacatccggctcaccgggtcggcggggcagtcgctgggcgcgttcctgccgcgcggcgtcacgctggagatggtgggcgacgccaacgactacgtcggcaagggcctgtccggcggccgcatcatcgtgcggccgcacccggacgcgacgttcgccgctgaacgtcaggtcatcgccggcaacacgctggcctacggcgccaccgcgggggagatgttcctgcgcgggcatgtgggcgagcggttctgcgtacgcaactcgggcgccaccgtcgtcgccgagggcgtgggcgaccacgccttcgaatacatgaccggtggccgtgccgtggtgctcggtccgaccggccgcaacctcgccgcgggcatgtccggcggtatcggctacgtcctcgacctcgaccagggcagcgtcaaccgcgagatggtcgagctgctcccgctcgagcccgaggatctgaactggttgaaggacatcgtgacccgtcaccacgaactcacccgctcggcggtcgccgcctcgctgctcggcgattggccgcgccggtcggcgagcttcacgaaggtcatgccgcgcgactacaagcgcgtgctggaggcgaccaaggccgcgaaggccgcgggccgcgacgtcgacgaggcgatcatggaggtggcgtctcgtggctga

SEQ ID NO: 2 - protein, 1524 aa, Amycolatopsis sp. ATCC 39116
  MISC_FEATURE (1)..(1524): Amino Acid Sequence of GltB
MIFSANPGKQGLYDPAMEQDSCGVAMVADIQGRRTHAIVTDGLTALINLDHRGAAGAEPTSGDGAGILVQLPDQLLREEAGFELPEPDAQGHHRYAAGIAFLPAEEEARGKAVALIERLADEESLEVLGWREVPVDADGADIGPTARSVMPHFAMLFVAGRPDAEGVRPSGLALDRLTFCLRKRVEHESVVAECGTYFPSLSSRTLVYKGMLTPEQLPAFFGDLRDPRLTSAIALVHSRFSTNTFPSWPLAHPFRFVAHNGEINTIRGNRNRMRAREALLESGLIPGDLTRLYPICSPEASDSASFDEVLELLHLGGRSLPHAVLMMIPEAWENHSTMDAQRRAFYQFHASLMEPWDGPACVTFTDGTLVGAVLDRNGLRPCRWWRTADDRVVLASEAGVLDVPPDQVVAKGRLKPGRMFLVDTEAGRIVADDEVKSELAKQHPYEEWLHAGLLQLADLQDRDHVTQSHDSVLRRQLAFGYSEEELKILLAPMAEKGAEPLGSMGTDTPPAVLSKRSRQLYDYFKQAFAQVTNPPLDAIREELVTSMSRIMGPERNLLDPGPASCRHIQLPYPVIDNDELAKLIHINDDGDLPGFACTVLSGLFEVDGGGKALAEAIERVRREASEAIAAGARTLVLSDRDSDHKMAPIPSLLLVSAVHHHLVRTKERLRVALVVETGDAREVHHIALLLGYGAAAVNPYLAFETIEDMIAQGAITGIEPRKAVRNYVNALVKGVLKIMSKMGISTVGAYTAAQVFESFGLSQELLDEYFTGTVSKLGGVGLDVLAEEVAVRHRRAYPDNPTDRVHRRLDSGGEYAYRREGELHLFTPETVFLLQHASKTRRDEVYRKYTEEVHRLSREGGALRGLFKFRKEGRAPVPIDEVESVESICKRFNTGAMSYGSISAEAHQTLAIAMNRIGGRSNTGEGGEDPERLYDPERRSAIKQVASGWFGVTSEYLVNADDIQIKMAQGAKPGEGGQLPPNKVYPWIARTRHSTPGVGLISPPPHHDIYSIEDLAQLIHDLKNANEQARIHVKLVSSLGVGTVAAGVSKAHADVVLISGHDGGTGASPLNSLKHAGTPWEIGLAETQQTLMLNGLRDRITVQVDGAMKTGRDVVVAALLGAEEYGFATAPLIVAGCIMMRVCHLDTCPVGVATQSPELRKRYTGQAEHVVNYFRFVAQEVRELLAELGFRTLDEAIGRADVLDTDDAVDHWKASGLDLSPIFQMPTDTPYGGARRKIREQDHGLEHALDRTLIQLSEAALEDAHPVRLELPVRNVNRTVGTLLGSEITRRYGGEGLPDGTIHIRLTGSAGQSLGAFLPRGVTLEMVGDANDYVGKGLSGGRIIVRPHPDATFAAERQVIAGNTLAYGATAGEMFLRGHVGERFCVRNSGATVVAEGVGDHAFEYMTGGRAVVLGPTGRNLAAGMSGGIGYVLDLDQGSVNREMVELLPLEPEDLNWLKDIVTRHHELTRSAVAASLLGDWPRRSASFTKVMPRDYKRVLEATKAAKAAGRDVDEAIMEVASRG

SEQ ID NO: 3 - DNA, 1509 bp, Amycolatopsis sp. ATCC 39116
  misc_feature (1)..(1509): Nucleic Acid Sequence of gltD
  misc_feature (1507)..(1509): stop codon
gtggctgaccccaagggcttcctgaagtacgagcgggtcgagccgcccaagcgccccaaggagcaccgcgccgaggactggcgcgaggtctacgtcgacctcgaaccggccgagcgcgaccagcaggtgcgcacccaggccacccgctgcatggactgcggcatcccgttctgccactcggccggttccggctgcccgctcggcaacctgatcccggagtggaacgacctggtgcgccgcggtgactggaccgcggccagcgaccggctgcacgccaccaacaacttcccggagttcaccgggaagctgtgcccggcgccgtgcgaggcgggctgcacgctgtccatctcgccgctgtccggcggcccggtcgcgatcaagcgcgtcgaggcgacgatcgcggagaagtcgtgggagctgggcctggcccagccgcaggtcgccgaggtggccagcggtcagcgcgtcgccgtggtcgggtccggcccggccggtctcgccgccgcccagcagctcacccgcgccgggcacgacgtgaccgtcttcgagcgggacgaccggctcggcgggctgctccgatacggcatccccgagttcaagatggagaagaagcacctcgacaagcgcctggcccagctcaagaaggagggcacgcagttcgtcacgggctgcgaggtgggcgtcgacatcaccgtcgaggagctgcgggcccgctacgacgcggtcgtgctcgccgtcggcgcgctgcgcggccgcgacgacaccaccacgcccggccgggagctcaagggcatccacctggcgatggagcacctggtgccggccaacaagcagtgcgagggcgacggcccgtcgccggtccacgcgcacggcaagcacgtggtgatcatcggcggtggtgacaccggcgccgactcctacggcaccgcgatccgccagggcgcggcctcggtggtccagctggaccagtacccgatgccgccgacgacccgcgacgacgagcggtcgccgtggccgacctggccgtacgtgctgcgcacctacccggcgcacgaggagggcggcgagcggaagttcggtgtcgccgtgcggcggttcgtgggcgacgagaacgggcacgtccgcgcgatcgagctgcagcaggtcaaggtcgtcaaggacccggagaccgggcgccgcgaggtgctgccggtgtcggacgagatcgaggagatcccggccgacctggtgctcttcgccatcgggttcgagggcgtggagcacatgcggctgctcgacgacctgggcatccggctgacccggcgcggcaccatctcgtgcggcccggactggcagaccgaggccccgggcgtgttcgtctgcggtgacgcccaccgcggcgcgtcgctggtcgtgtgggcgatcgcggagggccgctcggtggccaacgccgtcgacgcctacctgaccggcgcgtcggacctgccggccccggtgcatccgacggctctgccgctcgctgtggtgtaa

SEQ ID NO: 4 - protein, 502 aa, Amycolatopsis sp. ATCC 39116
  MISC_FEATURE (1)..(502): Amino Acid Sequence of GltD
MADPKGFLKYERVEPPKRPKEHRAEDWREVYVDLEPAERDQQVRTQATRCMDCGIPFCHSAGSGCPLGNLIPEWNDLVRRGDWTAASDRLHATNNFPEFTGKLCPAPCEAGCTLSISPLSGGPVAIKRVEATIAEKSWELGLAQPQVAEVASGQRVAVVGSGPAGLAAAQQLTRAGHDVTVFERDDRLGGLLRYGIPEFKMEKKHLDKRLAQLKKEGTQFVTGCEVGVDITVEELRARYDAVVLAVGALRGRDDTTTPGRELKGIHLAMEHLVPANKQCEGDGPSPVHAHGKHVVIIGGGDTGADSYGTAIRQGAASVVQLDQYPMPPTTRDDERSPWPTWPYVLRTYPAHEEGGERKFGVAVRRFVGDENGHVRAIELQQVKVVKDPETGRREVLPVSDEIEEIPADLVLFAIGFEGVEHMRLLDDLGIRLTRRGTISCGPDWQTEAPGVFVCGDAHRGASLVVWAIAEGRSVANAVDAYLTGASDLPAPVHPTALPLAVV

SEQ ID NO: 5 - DNA, 483 bp, Amycolatopsis sp. ATCC 39116
  misc_feature (1)..(483): Nucleic acid sequence of echR (repressor of ech-fcs operon)
  misc_feature (481)..(483): stop codon
gtggtgaccgaatcccgcgccgaggacgccccgctgaccctctacctggtcaagcggctggagctggtgatccgctcgctgatggacgacgcgctgcgcccgttcgggctgaccaccctgcagtacaccgcgctgaccgcgctgcggcaccgcaacgggctgtcgtccgcgcagctcgcgcgccgctcgttcgtccggccccagaccatgcacaccatggtgctcacgctggagaagtacgggctcatcgagcgcgcggaggacccggccaaccgccgggtcctgctcgccaccctcaccgagcgcggcaagcaggtcctcgacgagtgcacgccgctggtccgggagctcgaagaccggatgctctccggcatggacgacgaccgccgcgccgggttccgccgggacctggaggacggctacggcatgctcgcctcgcacgccaacgctcagcgcgcgttgacgaacggcggcggcgagtaa

SEQ ID NO: 6 - protein, 160 aa, Amycolatopsis sp. ATCC 39116
  MISC_FEATURE (1)..(160): Amino Acid Sequence of EchR (repressor of ech-fcs operon)
MVTESRAEDAPLTLYLVKRLELVIRSLMDDALRPFGLTTLQYTALTALRHRNGLSSAQLARRSFVRPQTMHTMVLTLEKYGLIERAEDPANRRVLLATLTERGKQVLDECTPLVRELEDRMLSGMDDDRRAGFRRDLEDGYGMLASHANAQRALTNGGGE